(19) United States
(12) Patent Application Publication
(10) Pub. No.: US 2015/0031998 A1
(43) Pub. Date: Jan. 29, 2015

(54) OBJECT INFORMATION ACQUIRING APPARATUS AND CONTROL METHOD THEREOF

(71) Applicant: CANON KABUSHIKI KAISHA, Tokyo (JP)

(72) Inventors: Hisako Kyono, Yokohama-shi (JP); Masakazu Taku, Machida-shi (JP)

(21) Appl. No.: 14/324,328

(22) Filed: Jul. 7, 2014

(30) Foreign Application Priority Data
Jul. 23, 2013 (JP) 2013-152643

(51) Int. Cl. A61B 8/00; A61B 5/00; A61B 8/08

(52) U.S. Cl. CPC A61B 8/403; A61B 8/0825; A61B 5/0095
USPC 600/437; 600/407

(57) ABSTRACT

Provided is an object information acquiring apparatus including: a holding unit that holds an object; a probe that receives an acoustic wave that propagates from the object; a housing that constitutes a holding container; a matching solution that is filled inside the holding container; and a pressure adjustment unit including a liquid quantity adjustment section that adjusts the quantity of the matching solution, and a distance adjustment section that adjusts the distance between the holding container and the object, wherein the pressure adjustment unit performs a first compression which increases a pressure applied to the object to a predetermined pressure value, and then performs a second compression which maintains the predetermined pressure value, while decreasing the liquid quantity inside the holding container.

### BACKGROUND OF THE INVENTION

1. Field of the Invention

The present invention relates to an object information acquiring apparatus and a control method thereof.

2. Description of the Related Art

An ultrasound inspection apparatus has been known as a measurement apparatus to acquire biological information. As the ultrasound inspection apparatus, an apparatus using an ultrasound echo and an apparatus using a photoacoustic effect are known. In order to acquire a good measurement result using such a measurement apparatus, it is necessary to hold a biological object (examinee) so that the biological object does not move. Further, in the case of diagnosing a breast or the like of an examinee using this apparatus, it is also demanded to reduce the burden imposed on the examinee during measurement as much as possible, and to not cause discomfort.

One method of holding a breast of an examinee so as not to move it is an immersion method, which performs measurement via an acoustic matching solution between the breast and a probe. As an example of the immersion method, Japanese Patent Application Laid-Open No. 2007-282960 discloses a compressing apparatus which maintains (keeps) a holding member constituted by an elastic film in an appropriate form in accordance with the size of the breast of the examinee. The compressing apparatus of Japanese Patent Application Laid-Open No. 2007-282960 adjusts the contact pressure between the breast and the film by adjusting the volume of liquid in a liquid encapsulating container, whereby a form of the film is maintained to be appropriate for imaging for various sizes of breasts.

Japanese Patent Application Laid-Open No. 2007-282960

### SUMMARY OF THE INVENTION

The apparatus disclosed in Japanese Patent Application Laid-Open No. 2007-282960 has a form appropriate for holding an object (breast) with an elastic film, and measuring the object using a probe which scans conically. In other words, the breast and the film can adhere to each other and the distance between the probe and the breast can be adjusted by adjusting the contact pressure between the breast and the film.

In order to measure an area up to a chest wall located at the base of the breast of the examinee, it is necessary to thinly stretched the breast, so that the distance between the probe to the chesty wall portion is not more than the length that the ultrasound wave can reach.

However, in the configuration disclosed in Japanese Patent Application Laid-Open No. 2007-282960, control of deformation of the film when the breast is held is insufficient. Therefore, depending on the shape or the size of the breast, a peripheral blind, where the film and the breast do not contact, may be generated. The generation of the peripheral blind interrupts the propagation of an acoustic wave, and becomes a cause of the probe not appropriately acquiring an acoustic wave. Further, if the deformation of the film cannot be sufficiently controlled, the holding member may protrude, which causes contact beteen the probe and the film.

In other words, in Japanese Patent Application Laid-Open No. 2007-282960, it is insufficient to balance stretching the breast as thin as possible and controlling the generation of the peripheral blind.

With the foregoing in view, it is an object of the present invention to control the generation of the peripheral blind while decreasing the thickness of the object, in an apparatus that holds an object using a film member.

The present invention provides an object information acquiring apparatus comprising:

a holding unit configured to hold an object;

a probe configured to receive, via the holding unit, an acoustic wave that propagates from the object held by the holding unit;

a housing configured to, along with the holding unit, constitutes a holding container;

a matching solution configured to be filled inside the holding container and to match acoustic impedance between the holding unit and the probe; and

a pressure adjustment unit including a liquid quantity adjustment section configured to adjust a quantity of the matching solution inside the holding container, and a distance adjustment section configured to adjust a distance between the holding container and the object, wherein

the pressure adjustment unit performs a first compression which increases a pressure applied to the object to a predetermined pressure value while adjusting the distance adjustment section without adjusting the liquid quantity adjustment section, and then performs a second compression which maintains the predetermined pressure value by decreasing the distance between the object and the holding container using the distance adjustment section, while decreasing the liquid quantity inside the holding container using the liquid quantity adjustment section.

The present invention also provides a control method of an object information acquiring apparatus that includes: a holding unit configured to hold an object; a probe configured to receive, via the holding unit, an acoustic wave that propagates from the object held by the holding unit; a housing configured to, along with the holding unit, constitute a holding container; a matching solution configured to be filled inside the holding container and to match acoustic impedance between the holding unit and the probe; and a pressure adjustment unit including a liquid quantity adjustment section configured to adjust a quantity of the matching solution inside the holding container, and a distance adjustment section configured to adjust a distance between the holding container and the object, the control method comprising the steps of:

causing the pressure adjustment unit to perform a first compression which increases a pressure applied to the object to a predetermined pressure value while adjusting the distance adjustment section without adjusting the liquid quantity adjustment section; and

causing the pressure adjustment unit to perform a second compression which maintains the predetermined pressure value by decreasing the distance between the object and the holding container using the distance adjustment section, while decreasing the liquid quantity inside the holding container using the liquid quantity adjustment section.

According to the present invention, in an apparatus that holds an object using a film member, the generation of the peripheral blind can be controlled while decreasing the thickness of the object.

Further features of the present invention will become apparent from the following description of exemplary embodiments with reference to the attached drawings.

### BRIEF DESCRIPTION OF THE DRAWINGS

FIG. 1 is a diagram depicting an operation according to Example 1 (initial position);

FIG. 2 is a diagram depicting an operation according to Example 1 (first compression);

FIG. 3 is a graph depicting the time-based changes of compressing load and compressing distance;

FIG. 4 is a diagram depicting an operation of Example 1 (second compression);

FIG. 5 is a flow chart depicting a drive control method according to Example 1;

FIG. 6 is a diagram depicting an arrangement according to Example 2;

FIG. 7 is a flow chart depicting a drive control method according to Example 2;

FIG. 8 is a diagram depicting an operation according to Example 2 (first compression); and

FIG. 9 is a diagram depicting an operation according to Example 2 (second compression).

### DESCRIPTION OF THE EMBODIMENTS

Preferred embodiments of the present invention will now be described with reference to the drawings. Dimensions, materials and shapes of composing elements described hereinbelow and relative positions thereof should be appropriately changed in accordance with a configuration and various conditions of an apparatus to which the invention is applied, and are not intended to limit the scope of the invention by the following description.

An object information acquiring apparatus of the present invention includes an apparatus that receives an acoustic wave, which is generated by a photoacoustic effect in an object to which light (electromagnetic wave) is irradiated, and is propagated, and acquires the characteristic information of the object information as image data. The characteristic information acquired at this time indicates the generation source distribution of an acoustic wave generated by light irradiation, the initial sound pressure distribution in the object, the light energy absorption density distribution and absorption coefficient distribution derived from the initial sound pressure distribution, and the concentration distribution of a substance which constitutes a tissue, for example. The concentration distribution of a substance is, for example, oxygen saturation distribution or oxyhemoglobin/deoxyhemoglobin concentration distribution.

The object information acquiring apparatus of the present invention includes an apparatus that uses an ultrasound echo technology that transmits an ultrasound wave to an object, receives a reflected wave (echo wave) which is reflected and propagated inside the object, and acquires the object information as image data. The characteristic information acquired at this time is information that reflects differences of acoustic impedance in the tissue inside the object.

The present invention is also suitable for a breast holding apparatus that holds a breast in a state of being stretched thinly and acquires information inside the breast. The present invention can also be regarded as a method for controlling the object information acquiring apparatus, and a program that allows an information processor to execute the control method.

The acoustic wave in the present invention is typically an ultrasound wave, including such elastic waves as a sound wave, an ultrasound wave and an acoustic wave. In photoacoustic tomography, an acoustic wave generated by the photoacoustic effect is called a “photoacoustic wave” or a “light-induced ultrasound wave”.

### Example 1

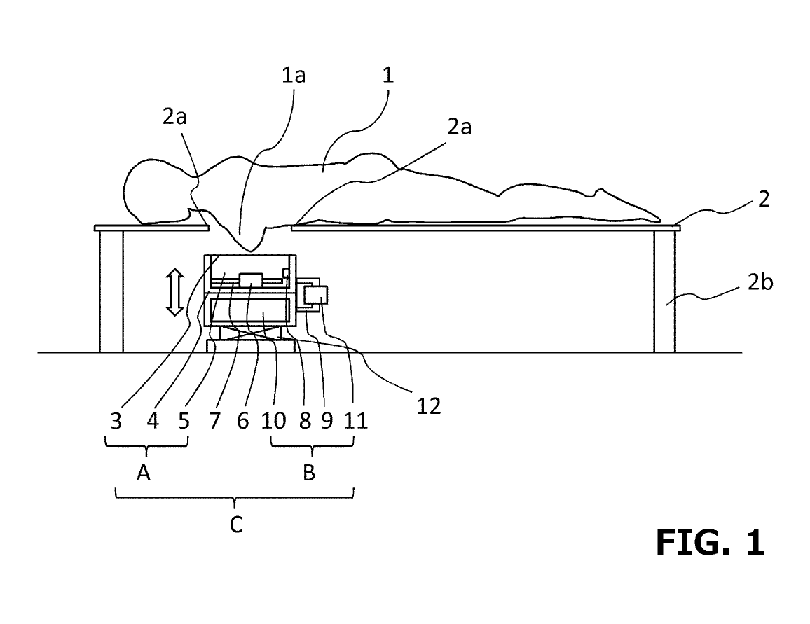

Embodiments of the present invention will now be described in detail with reference to the drawings. FIG. 1 to FIG. 3 are diagrams depicting an operation according to Example 1 of the present invention.

<Configuration of Apparatus>

An inspection target is a breast la, which is a part of examinee 1. The examinee 1 lies on a bed 2 face down (prone position). The bed 2 includes an opening 2a to insert the breast 1a, and legs 2b to maintain height.

(Holding Container)

A holding member 3 holds the breast 1a. For the holding member, it is preferable to select a material of which acoustic impedance is close to that of a human body. Thereby the reflection on an interface between the breast 1a and the holding member can be suppressed, and detection of an ultrasound wave signal improves. The thickness of the holding member 3 is preferably ¼ or less of the wavelength of the ultrasound wave. Thereby the reflection of the ultrasound wave by the holding member can be prevented, which decrease harmful noise. As a consequence, the holding member 3 is preferably thin, and typically a film member is used for the holding member 3. The holding member 3 corresponds to the holding unit of the present invention.

A material suitable for the holding member 3 is, for example, polymethylpentene or silicone rubber. By using a flexible elastic material as the holding member 3, the holding member 3 can be deformed in accordance with the shape of the breast 1a.

A frame member 4 has a rectangular parallelepiped shape of which top surface has an opening. The holding member 3 is attached to the top surface of the frame member 4 so that the holding member 3 and the frame member 4 constitute a water tight structure. The frame member 4 must have a strength to compress the breast 1a. The frame member corresponds to the housing of the present invention.

An acoustic matching solution 5 fills the water tight structure of the holding member 3 and the frame member 4. For the acoustic matching solution 5, a material of which acoustic impedance is close to that of a human body and of which attenuation of the ultrasound wave is small, such as water or oil, must be selected.

A holding container A is one unit which encloses the water tight structure constituted by the holding member 3 and the frame member 4, and is filled with the acoustic matching solution 5. The breast 1a is compressed by the elevation of a holding unit C, including the holding container A. It is preferable to operate the holding unit C in steps for the compressing operation, in terms of pressure adjustment and ease of control. One step in this case may be called a “compression stroke”. The present invention, however, can also be implemented by continuously operating the holding unit C.

(Ultrasound Transmitting/Receiving Mechanism)

The ultrasound probe 6 has an element that transmits an ultrasound wave, receives an ultrasound wave, and converts it into electric signals.

A scanning mechanism 7 is a mechanism that operates the ultrasound probe 6 for scanning in the holding container A. The scanning mechanism 7 allows the ultrasound probe 6 to scan a surface in parallel with the holding member 3, so as to measure the entire surface of the breast 1a.

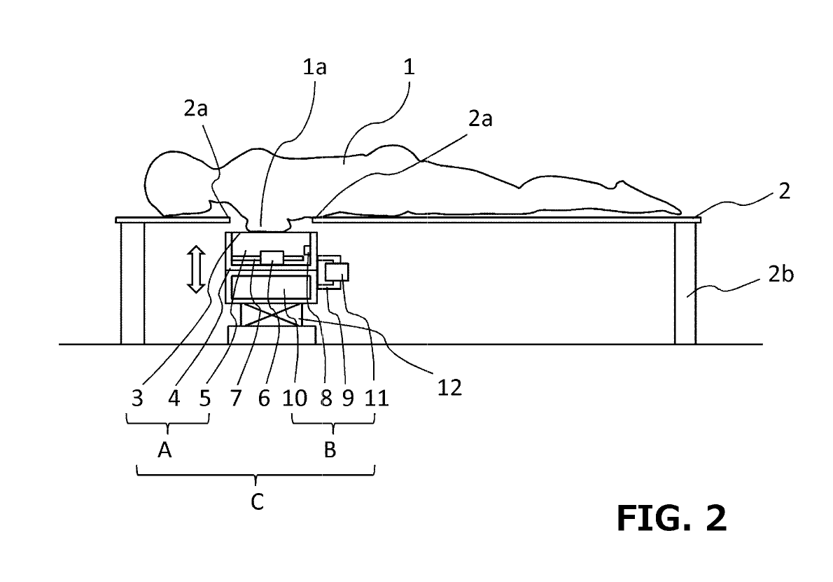

If there is an air layer between the ultrasound probe 6 and the breast 1a, the ultrasound wave reflects on the interface between the air and the breast 1a due to the difference in acoustic impedance therebetween, hence the detectability of the ultrasound signal decreases and the S/N ratio drops. Therefore the attenuation of the ultrasound wave can be reduced by the ultrasound probe 6 scanning inside the holding container A filled with the acoustic matching solution 5 as shown in FIG. 1 and FIG. 2. Scanning without contacting the breast 1a and the ultrasound probe 6 also becomes possible.

(Liquid Quantity Adjustment Unit)

A liquid quantity adjustment unit B will be described next. The liquid quantity adjustment unit B is constituted by a pressure detection unit 8, a supply/discharge pipe 9, a tank 10 and a pump 11.

The pressure detection unit 8 is installed inside the holding container A, and detects the pressure inside the holding container A.

The pump 11 moves the acoustic matching solution between the tank 10 and the holding container A via the supply/discharge pipe 9. Thereby the water quantity (quantity of the acoustic matching solution) inside the holding container A is adjusted. The liquid quantity adjustment is controlled by a control circuit (not illustrated) according to the detection result by the pressure detection unit 8. The liquid quantity adjustment unit corresponds to the liquid quantity adjustment section of the present invention.

The liquid quantity adjustment is required for the second compression of the present invention, although details thereof will be described later. In the second compression, the holding member 3 is pressed against the breast 1a along the periphery of the breast 1a, while maintaining the water pressure inside the holding container A constant, as set in the first compression, which is the conventional compression.

In the liquid quantity adjustment during the second compression, if the breast is relatively small, the compression is stopped when the holding unit C reaches the upper limit of the compression stroke, or when the deformation amount of the holding member 3 reaches or exceeds a predetermined amount. If the breast 1a is relatively large, the second compression is stopped when the pressure control inside the holding container A, due to the liquid quantity adjustment inside the holding container A, is disabled.

The holding container A is integrated with the ultrasound probe 6, the scanning mechanism 7 and the liquid quantity adjustment unit B, which are enclosed in the holding container A, and constitute the holding unit C.

(Compression Mechanism)

An elevation mechanism 12 moves the holding unit C in the vertical direction, so that the holding member 3 compresses the breast 1a. An example of measuring the compressing force applied to the breast 1a is attaching a load sensor to the elevation mechanism 12. In this case, the load sensor measures not only the compressing force, but also the weight of the holding unit C. However the compressing force can be measured by subtracting the weight of the holding unit C from the detected value, since the weight of the holding unit C is designed not to change even if the liquid quantity inside the holding container A is adjusted. The elevation mechanism corresponds to the distance adjustment section of the present invention. The elevation mechanism and the liquid quantity adjustment unit may collectively be regarded as the pressure adjustment unit, since the pressure applied to the object can be adjusted either by adjusting the quantity of the matching solution or by adjusting the distance between the object and the holding unit.

The elevation mechanism 12 is moved up or down by a motor or manually. For example, FIG. 1 shows a pantograph type elevation mechanism 12 which is driven by an electric motor, for example. In the case of manual driving, the elevation mechanism is moved up/down by operating a handle where a deceleration mechanism and a one-way mechanism for fall prevention are provided.

(Compression Procedure)

In the initial position in FIG. 1, the pressure value inside the holding container A is set to be the atmospheric pressure or more (initial pressure), and the holding member 3 is set to be substantially flat or a convex shape protruding toward the object. Thereby the breast 1a is pressed by an approximately flat surface, and the breast 1a can be deformed to be flat.

FIG. 2 shows a first compression state after the holding unit C is moved up for a required number of strokes by the elevation mechanism 12 from the state in FIG. 1. The quantity of the acoustic matching solution 5 inside the holding container A is not changed until this stage.

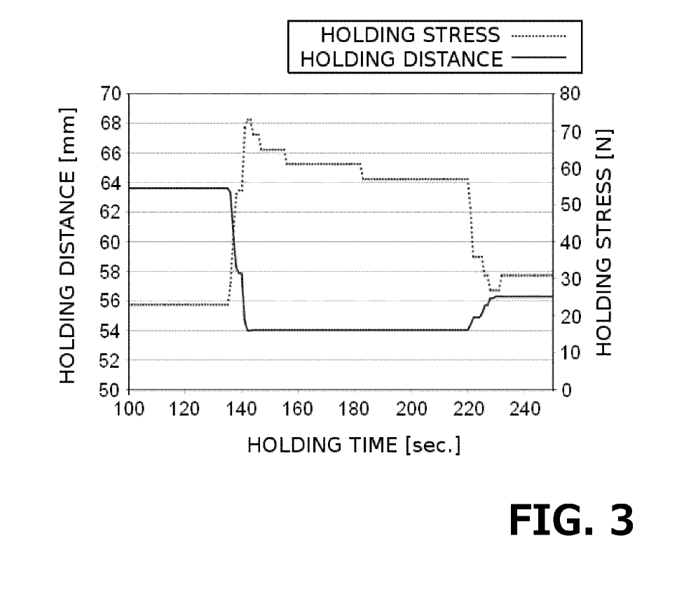

FIG. 3 is a graph depicting a time-based change of the distance between two holding plates and the compressing force when the breast 1a is compressed by the two holding plates. As this graph shows, the pressing force that is applied to the examinee 1 is decreased by about 10N in about 10 seconds from the peak of the compressing force. This is probably because the compressing force applied to the breast 1a is decreased by pausing the elevation of the holding unit C for several seconds after the first compression, hence the burden imposed on the examinee 1 is reduced. In this way the period when the compression state is not changed can be about several seconds, such as 10 seconds or less.

This change in compressing force can occur in the same manner in a system using the holding unit shown in FIG. 1. Therefore in the present invention as well, the breast can be deformed to be flat while reducing the burden imposed on the examinee 1 by the first compression. As a result, the distance between the chest wall of the examinee 1 to the holding member 3 can be minimized, and the S/N ratio in the entire breast can be improved.

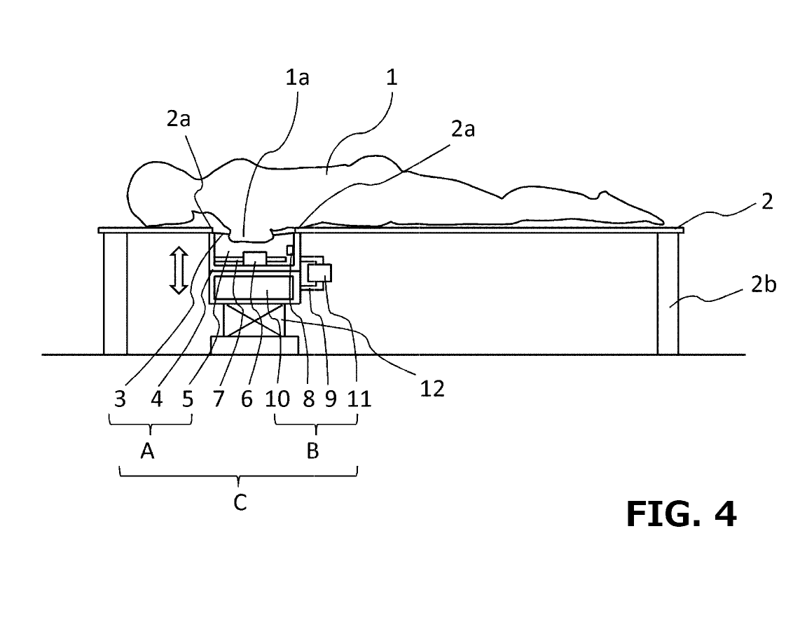

FIG. 4 shows the state of the second compression. In the second compression, the breast 1a is compressed while adjusting the water pressure inside the holding container A. Then the holding member 3 can be deformed by the resilience of the breast 1a which is generated by elevating the holding member 3, so that the holding member 3 and the peripheral portion of the breast 1a can adhere to each other. As a consequence, the distance between the chest wall of the examinee 1 and the holding member 3 can be minimized, and a blind in the peripheral portion can be reduced.

<Processing Flow>

In the ultrasound diagnostic apparatus having the above configuration, a drive control method, for decreasing the distance to the chest wall and increasing the adhesiveness between the breast 1a and the holding member 3 in a state where the burden imposed on the examinee 1 is reduced, will be described using an example.

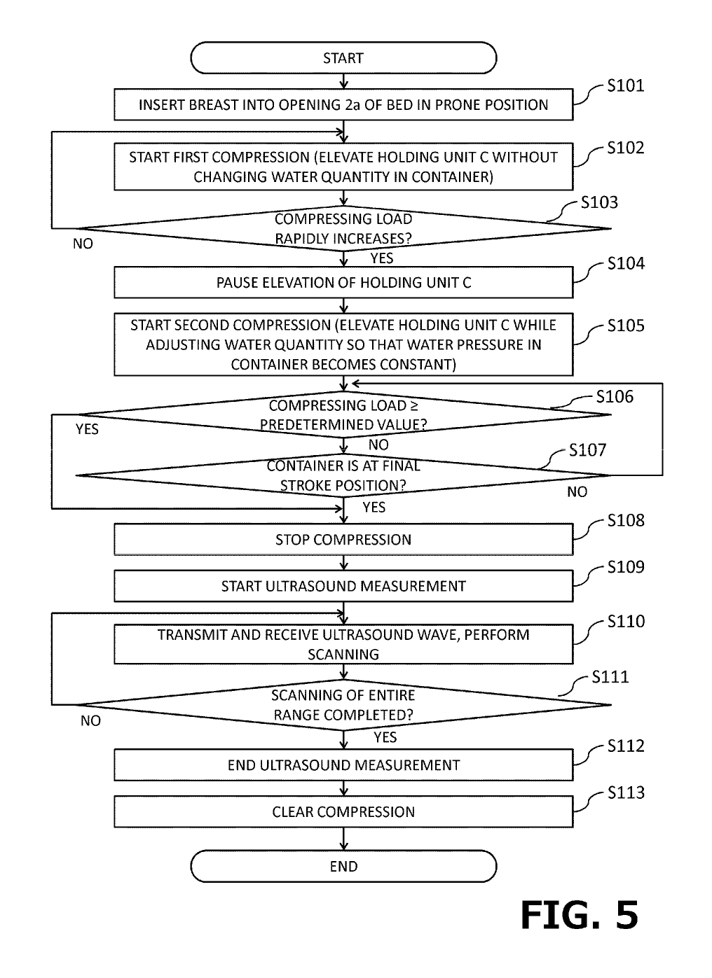

FIG. 5 is a flow chart depicting a drive control method according to the present invention.

In S101, the examinee 1 is lying on a bed face down as shown in FIG. 1, and the breast 1a is inserted into the opening 2a.

Then in S102, the first compression is performed as shown in FIG. 2, where the breast 1a is compressed by elevating the holding unit C without adjusting the quantity of the acoustic matching solution 5 inside the holding container A. This example is called a “single-sided holding type”, since the compressing force is applied to the breast by the holding member from one side of the breast.

During compression in S103, it is checked whether the compressing force measured by the elevation mechanism 12 increases rapidly with respect to the compressing distance. If a rapid increase is not observed (NO in S103), the first compression is continued.

If the compressing force increases rapidly (YES in S103), processing advances to S104, and the first compression is ended and the holding unit C is paused for several seconds. During the check of the increase in the compressing force in S103, differentiation of the change of the compressing force with respect to the compressing distance is also calculated, and processing advances to S104 when a predetermined amount of increase from the initial change is observed.

Then in S105, the second compression shown in FIG. 4 is performed. In the second compression, the breast 1a is compressed by elevating the holding unit C while keeping the pressure inside the holding container A constant. The pressure value is adjusted by changing the quantity of the acoustic matching solution 5 while being measured by the pressure detection unit 8. By appropriately adjusting the quantity of the acoustic matching solution, an area where the breast and the holding member (film) do not contact can be decreased. As a result, a peripheral blind can be suppressed, and in the subsequent ultrasound transmission/reception processing, good characteristic information can be acquired over a wide range inside the object.

In S106, the second compression is continued, and whether the compressing load has reached or exceeded a predetermined value is detected. If the predetermined compressing force is exceeded (YES in S106), compression is stopped in S108.

The predetermine compressing force is determined in terms of the distance between the breast 1a and the ultrasound probe, the deformation amount of the holding member 3, the prevention of contact between the holding member 3 and the ultrasound probe 6, reducing the burden imposed on the examinee, and the size of the breast. It is preferable that the breast is compressed to be as thin as possible in terms of improving the S/N ratio, but this increases the discomfort of the examinee. Further, if the breast is large, the deformation amount of the holding member 3 is also large, therefore the holding member 3 may contact with the ultrasound probe 6, which may increase the discomfort of the examinee, and cause an alignment error of the apparatus. Therefore in this step, compression is stopped when the deformation amount exceeds the tolerance.

For the predetermined compressing force, a value according to the respective breast size may be stored to and read from a storage device in advance. The operator may input a value using an input device (not illustrated) while observing the compressing and holding states. A compression stopping unit for use by the operator or the examinee may be provided.

If the second compression does not exceed the predetermined compressing force (NO in S106), and the holding unit C reaches the final stroke position (YES in S107), processing advances to S108 and elevation stops. By the procedure thus far, the breast 1a is held in an appropriate position. The position of the final stroke is set such that the compression on the breast does not exceed a predetermined pressure value, and may be different from individual to individual depending on the size of the breast. The position of the final stroke may also be determined by the physically movable range of the holding unit, the specified measurement area and the image quality.

Then processing advances to S109, and the ultrasound measurement is started.

Then in S110, scanning of the imaging target range is started while transmitting/receiving ultrasound waves.

In S111, it is checked whether the scanning completed or not. This is a check on whether measurement is completed for all the scanning areas which are set in advance.

If the scanning ends (YES in S111), processing advances to S112 and the measurement ends. Then processing advances to S113, and compression on the breast 1a is cleared.

As described above, according to this example, when the breast is compressed and held by the film member of the container filled with the acoustic matching solution, the contact between the breast and the film can be optimized with a minimal peripheral blind, while stretching the breast as thin as possible.

### Example 2

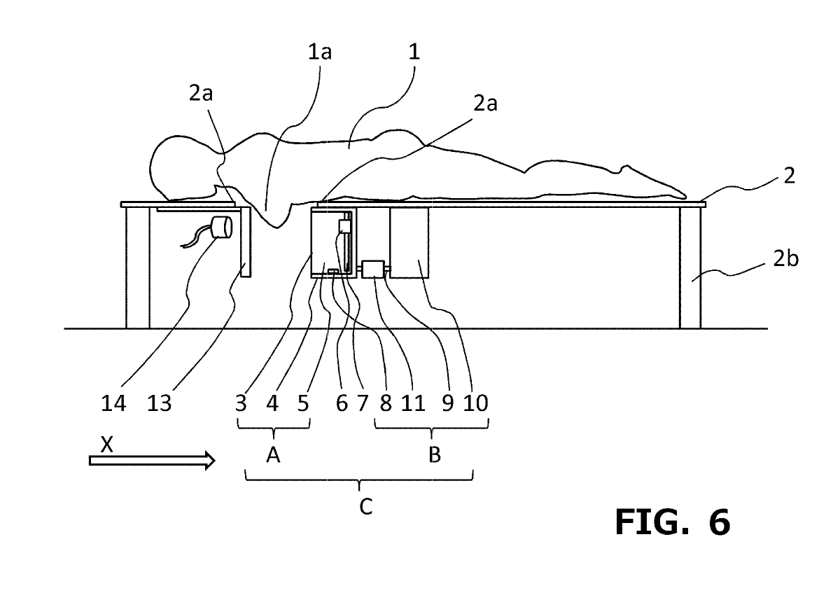

In Example 1, the single-sided holding type ultrasound echo apparatus was described. In Example 2, on the other hand, a double-sided holding type apparatus utilizing a photoacoustic effect will be described with reference to FIG. 6 to FIG. 9. The apparatus of this example holds the breast 1a using a holding member that is disposed in the cephalocaudal direction of the examinee.

<Configuration of Apparatus>

FIG. 6 is a diagram depicting an operation of this example. The double-sided holding type apparatus is constituted by a bed 2, a holding unit C (holding member 3, frame member 4, acoustic matching solution 5, ultrasound probe 6, scanning mechanism 7, pressure detection unit 8, supply/discharge pipe 9, tank 10 and pump 11), a light source 14 and a movable compressing plate 13. Primarily the differences from Example 1 will be described.

In an apparatus utilizing a photoacoustic effect, light emitted from the light source 14 is irradiated onto the breast 1a and an absorber inside the breast 1a absorbs the light energy, whereby the absorber expands and contracts. The acoustic wave generated at this time is detected by the ultrasound probe 6, and the detected signals are converted into an image. The wavelength of the light emitted from the light source 14 is preferably 600 nm to 900 nm of near-infrared rays, since light absorption from water, which is the major component of the tissue inside an organism, is small, and the absorption coefficient spectrum of oxyhemoglobin or deoxyhemoglobin presents a characteristic form if this wavelength is used.

The movable compressing plate 13 is a plate member which compresses the breast 1a inserted between this plate and the holding unit C. Typically the movable compressing plate 13 has substantially a flat form. Preferably the movable compressing plate 13 is made from a material which can transmit light from the light source, and which is rigid enough not to deform very much when the breast 1a is compressed to be flat (e.g. acrylic). In the photoacoustic apparatus, a mechanism (not illustrated) to move the movable compressing plate 13 in the X direction and a sensor (not illustrated) to measure the compressing force are also disposed along with the movable compressing plate 13.

In the holding unit C, a moving mechanism (not illustrated), to adjust the position of the holding unit and to fix the holding unit in an appropriate position with respect to the breast, is also disposed.

In this example, the light is irradiated from the movable compressing plate 13 side. However the light source may be disposed on the side of the ultrasound probe 6, which is the light receiving unit. In this case, for the holding member 3, it is necessary to select a material that transmits light with a 600 nm to 900 nm wavelength which is the spectrum of near-infrared rays. An appropriate material that satisfies this condition is a transparent material made of polymethylpentene or silicone rubber.

<Processing Flow>

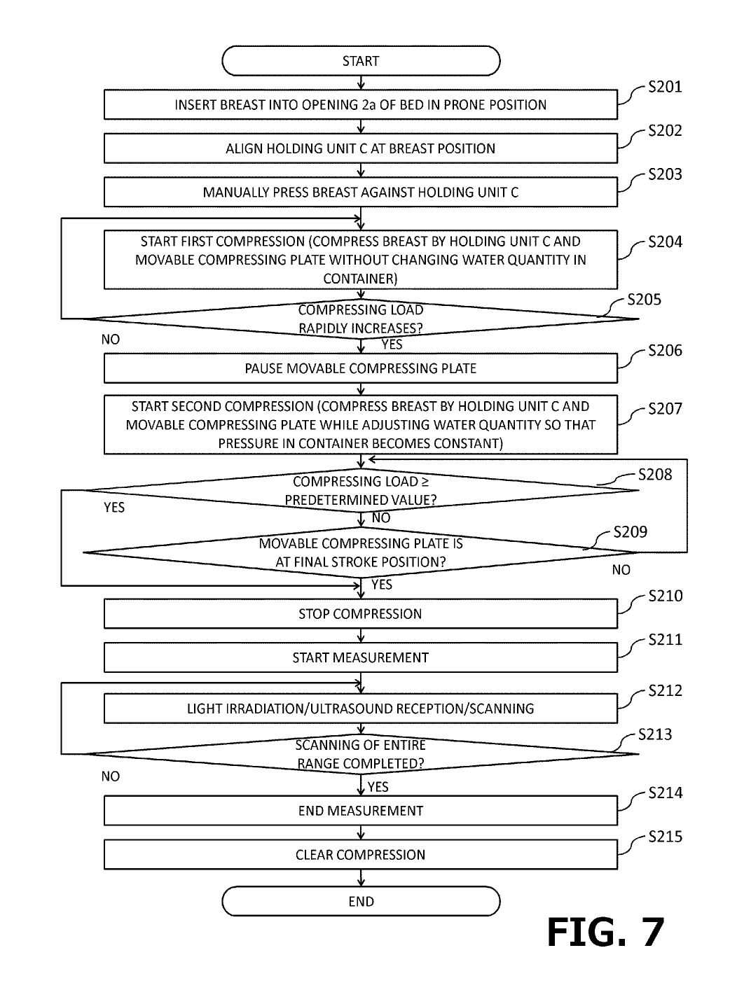

FIG. 7 is a flow chart depicting a drive control method of the present invention. Primarily the differences from FIG. 5 will be described.

S201 is the same as S101 in FIG. 5.

Then in S202, the holding unit C is set to an appropriate position. A position where the holding member 3 comes close to a bottom end (surface on the feet side of the examinee) of the breast is suitable.

In S203, the physician or the technician manually presses the breast 1a against the holding member 3.

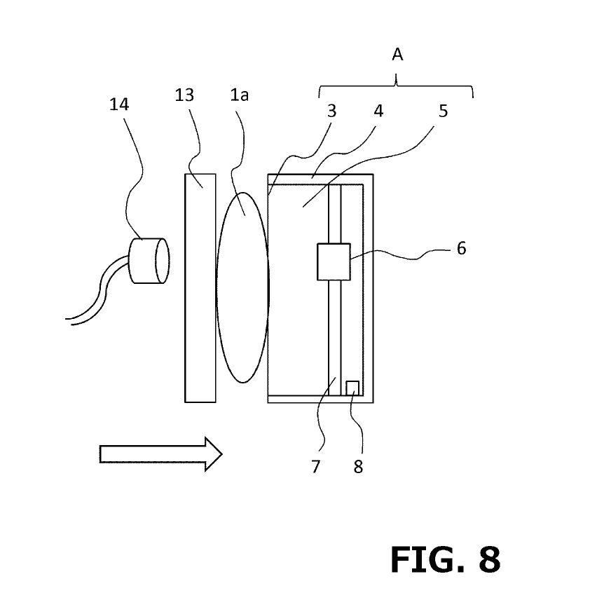

In S204, the movable compressing plate 13 is moved toward the holding unit C and the first compression is started. FIG. 8 is a diagram depicting an operation of the breast 1a, the holding container A, which is a part of the holding unit C, and the movable compressing plate 13 during the first compression. The first compression performed here is the same as Example 1, except that the movable compressing plate 13, instead of the holding unit C, is moved.

In the first compression, it is checked whether the compressing force increases rapidly with respect to the compressing distance (S205), and the movable compressing plate 13 is paused for several seconds if the compressing force increases rapidly (S206), which is the same as S103 and S104 in FIG. 5.

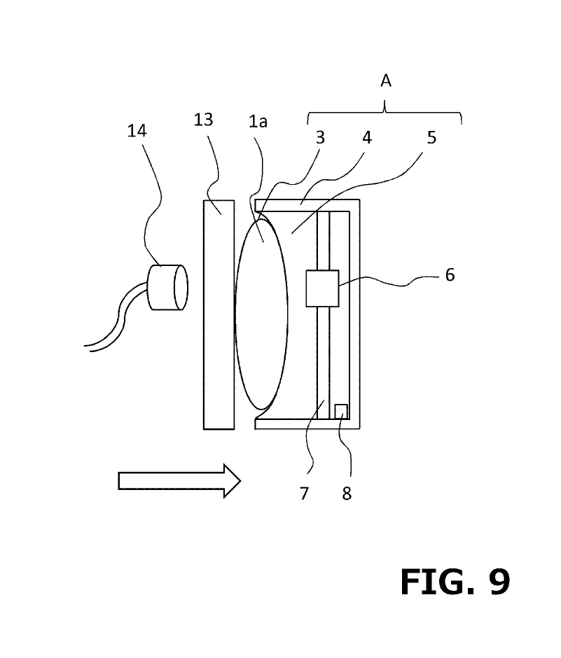

Then in S207, the second compression is performed, where the movable compressing plate 13 is moved while adjusting the quantity of the acoustic matching solution, so that the water pressure value becomes constant. FIG. 9 is a diagram depicting the operation during the second compression.

The compressing load detection in S208 and the stroke position detection in S209 are the same as S106 and S107 in FIG. 5.

When the compression is stopped in S210, the final stroke position in Example 1 (S108 in FIG. 5) is a position where the holding unit C stops just before contacting with the bed 2. In Example 2, the final stroke position is a position where the holding unit C stops just before contacting the movable compressing plate 13. By the procedure thus far, the breast 1a is held at an appropriate position.

Then in S211, the photoacoustic measurement is started.

In S212, light is irradiated from the light source 14, and a measurement target range is scanned while receiving the generated ultrasound wave by the probe.

When scanning ends (YES in S213), processing advances to S214, and the measurement ends. Then processing advances to S215, and compression of the breast 1a is cleared.

By using a light-transmissive thin film for the holding member 3 and disposing the light source 14 in the holding container A, the present invention can also be applied to an apparatus using the photoacoustic effect. In Example 2, the double-sided holding type breast compression is applied to the photoacoustic apparatus, but the double-sided holding type can also be applied to an apparatus using ultrasound echoes.

By appropriately setting the position of the light source and the light transmissivity of the holding member (film), the present invention can also be applied to the single-sided holding type photoacoustic apparatus.

As described above, according to this example as well, when the breast is compressed and held by the film member of the container filled with the acoustic matching solution, the contact between the breast and the film can be optimized with a minimal peripheral blind, while stretching the breast as thin as possible. Thereby the distance between the chest wall and the holding member can be decreased considerably, and the S/N ratio can be improved.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-152643, filed on Jul. 23, 2013, which is hereby incorporated by reference herein in its entirety.

What is claimed is:

1. An object information acquiring apparatus comprising:
a holding unit configured to hold an object;
a probe configured to receive, via the holding unit, an acoustic wave that propagates from the object held by the holding unit;
a housing configured to, along with the holding unit, constitutes a holding container;
a matching solution configured to be filled inside the holding container and to match acoustic impedance between the holding unit and the probe; and
a pressure adjustment unit including a liquid quantity adjustment section configured to adjust a quantity of the matching solution inside the holding container, and a distance adjustment section configured to adjust a distance between the holding container and the object, wherein
the pressure adjustment unit performs a first compression which increases a pressure applied to the object to a predetermined pressure value while adjusting the distance adjustment section without adjusting the liquid quantity adjustment section, and then performs a second compression which maintains the predetermined pressure value by decreasing the distance between the object and the holding container using the distance adjustment section, while decreasing the liquid quantity inside the holding container using the liquid quantity adjustment section.

2. The object information acquiring apparatus according to claim 1, wherein
the pressure adjustment unit ends the first compression at a point when the pressure value rapidly increases with respect to a compressing distance in the first compression.

3. The object information acquiring apparatus according to claim 1, wherein
the pressure adjustment unit has a period of not changing the compression state after the first compression and before starting the second suppression.

4. The object information acquiring apparatus according to claim 3, wherein
the period of not changing the compression state is 10 seconds or less.

5. The object information acquiring apparatus according to claim 1, wherein
the holding unit is formed of a film type elastic material.

6. The object information acquiring apparatus according to claim 5, wherein
the holding unit has a thickness not exceeding ¼ of the wavelength of the acoustic wave.

7. The object information acquiring apparatus according to claim 1, wherein
the liquid quantity adjustment section adjusts the liquid quantity before the first compression so that the holding unit becomes substantially flat or protrudes toward the object.

8. The object information acquiring apparatus according to claim 1, wherein
the probe transmits an acoustic wave to the object, and receives the acoustic wave reflected by the object.

9. The object information acquiring apparatus according to claim 1, wherein
the acoustic wave is a photoacoustic wave which is generated when the object is irradiated with light.

10. A control method of an object information acquiring apparatus that includes: a holding unit configured to hold an object; a probe configured to receive, via the holding unit, an acoustic wave that propagates from the object held by the holding unit; a housing configured to, along with the holding unit, constitute a holding container; a matching solution configured to be filled inside the holding container and to match acoustic impedance between the holding unit and the probe; and a pressure adjustment unit including a liquid quantity adjustment section configured to adjust a quantity of the matching solution inside the holding container, and a distance adjustment section configured to adjust a distance between the holding container and the object, the control method comprising the steps of:
causing the pressure adjustment unit to perform a first compression which increases a pressure applied to the object to a predetermined pressure value while adjusting the distance adjustment section without adjusting the liquid quantity adjustment section; and
causing the pressure adjustment unit to perform a second compression which maintains the predetermined pressure value by decreasing the distance between the object and the holding container using the distance adjustment section, while decreasing the liquid quantity inside the holding container using the liquid quantity adjustment section.
